(19) United States
(12) Patent Application Publication
(10) Pub. No.: US 2021/0379219 A1
(43) Pub. Date: Dec. 9, 2021

(54) PORTABLE SANITIZING ARRANGEMENT

(71) Applicant: Safer Planet Inc., Millbrae, CA (US)

(72) Inventors: Abdi Hariri, Hillsborough, CA (US); Marco Mora, Millbrae, CA (US); Jeffrey Brooks, Escondido, CA (US)

(21) Appl. No.: 17/338,622

(22) Filed: Jun. 3, 2021

Related U.S. Application Data
(60) Provisional application No. 63/158,253, filed on Mar. 8, 2021.
(60) Provisional application No. 63/036,071, filed on Jun. 8, 2020.

(51) Int. Cl. A61L 2/10; A61L 2/26

(52) U.S. Cl. CPC A61L 2/10; A61L 2202/20; A61L 2202/16; A61L 2/26

(57) ABSTRACT

A portable sanitizing arrangement is disclosed comprising essentially a protective case for a mobile productivity device such as a smartphone together with an ultraviolet light source for convenient disinfecting of high-touch objects and surfaces. The arrangement further comprises user controls and interfaces to enable the ultraviolet light source to safely emit light at wavelengths known to kill and destroy bacterial and viral species among other potentially harmful microorganisms. The sanitizing arrangement may irradiate the mobile productivity device itself as well as commonly encountered objects and surfaces in work, travel, entertainment and home environments with substantially greater ease and convenience and at potentially lower cost than with known sanitizing devices and methods.

### CLAIM OF PRIORITY

This application claims priority to U.S. Provisional Patent Application No. 63/036,071, filed Jun. 8, 2020. This application also claims priority to U.S. Provisional Patent Application No. 63/158,253 filed Mar. 8, 2021. Both above-identified applications are herein incorporated by reference.

### 1. FIELD OF THE INVENTION

The present invention relates to apparatus and methods for sanitizing, that is, disinfecting, objects and surfaces that one may encounter in one's daily activities and environments.

### 2. DESCRIPTION OF THE RELATED ART

Recently, public interest in personal hygiene and sanitizing and maintaining sanitary objects and surfaces encountered at work, home and in public spaces has soared due to the outbreak of the novel coronavirus and the associated Covid-19 disease pandemic, also referred to as SARS CoV-2. However, SARS CoV-2 is just one of many pathogenic human disease vectors that have existed at any given time, including but not limited to SARS, MERS, E. Coli, Staphylococcus, Ebola, and the H1N1 influenza outbreaks. Other disease vectors also may include certain species of fungi and protozoa. In general, disease vectors are spread by either direct or indirect contact, or by speaking, coughing or sneezing actions that produce microscopic aerosols that in turn deposit such species onto external surfaces or objects. Some species remain viable for long periods of time extending up to many days and may result in further transmission of the species, for example whereby a person touches a contaminated surface or object and subsequently touches that person's mouth, nose or eyes, or whereby the first person may touch another person who then in turn touches his or her mouth, nose or eyes. Depending on the species of pathogen, one transmission mode may dominate over another. For example in North American hospitals, so-called Hospital Acquired Infections (HAI) account for 5-30% of all infections, and the dominant transmission method is believed to be from contaminated surfaces. Additionally, the emergence of “super bugs”, i.e., pathogenic species that are resistant to therapeutics by mutation) increase the need for alternative strategies to help prevent infectious microorganisms from entering one's body.

Ultraviolet light has been long known to be an effective disinfecting agent. As shown in FIG. 1, ultraviolet (UV) light, comprising a wavelength range of 100-400 nanometers (nm), naturally occurs in the electromagnetic spectrum emitted by our sun. However, UV light also can be generated by incandescent lamps, lasers, plasmas, electrical gas discharges, and more recently by solid state light emitting diodes (LEDs). As further shown in FIG. 1, UV light further may be subdivided into so-called UVA light defined as ranging from about 315-400 nm in wavelength, UVB light defined as ranging from about 280-315 nm, and UVC light defined as ranging from about 200-280 nm. There is a fourth category of UV light below 200 nm that only propagates in vacuum and therefore for obvious reasons is called vacuum ultraviolet light (VUV); however that band of UV light is not applicable to the present invention and requires no further explanation.

In FIG. 2 representative antimicrobial effect of UVC light is illustrated (source Xylem Inc.). It is theorized that bacteria and viruses are inactivated because they consist of micrometer or smaller dimension, and therefore UVC light is able to penetrate the entirety of the organisms and thereby inactivate their reproductive structures.

UV light sources that are effective in neutralizing and/or destroying bacteria and/or viruses are commercially available, for example the large, autonomous wheeled LightStrike™ “germ-zapping robots” manufactured by Xenex Disinfection Services of San Antonio, Tex., USA. Xenex robots emit broadband UV light ranging from 200-315 nm and are intended for industrial or clinical use at night or when patients and staff are not present. In another application, narrow band UV light produced by low pressure mercury lamps for many years has been used to sanitize water for drinking, commercial and industrial applications as described by Philips Lighting BV of Eindhoven, Netherlands (Philips publication 3222 635 61801, November 2006).

However, such UV sanitation systems are large, complex and expensive devices and embody form factor, power and weight considerations that cannot conveniently be carried or used outside of a laboratory, clinic, or industrial setting.

Another consideration is that long exposure to broadband UV light, and more specifically UVB light, is associated with sunburn and elevated risk of cancer in humans and animals. But, recent research by Miwa et al. suggests that UVC is a useful tool for the treatment of certain cancer cells to a depth of about 40 μm without damage to deep issue. Additionally, far UVC light comprising shorter wavelengths from about 200-230 nm recently has been shown to not harm mammalian skin tissue (e.g., Buonanno et al. detected no adverse reaction to moderate-to-low dose exposure of human tissue to 207-222 nm far UVC light. Buonanno et al. theorize that no harm results because far UVC light at wavelengths below about 230 nm is characterized by strong absorbance of the shorter wavelengths in biological materials and therefore cannot penetrate even the outer layers of human skin or eye tissues. The testing and results of Buonnano et al. are discussed in more detail below.

In general, any reaction of mammalian skin and/or eye tissue to UVC light will be a function of total dose which may be defined by the energy of the incident UVC light, the absorbance of such light by the tissue, and the cumulative exposure time. It is expected that further studies and characterization of safe UVC dose, especially far UVC, will be forthcoming.

On the other hand, any of various known brands of conventional disposable sanitary wipes can be used to wipe surfaces of objects that one intends to handle. However, sanitary wipes typically embody considerable moisture comprising alcohol or other disinfectant chemicals such as quaternary ammonium compounds that may be incompatible with or harmful to electronic devices, sensitive human respiratory systems and skin, and fabrics, among other things. Also, depending on chemical content, wipes may not work on a broad class of microbes and improper application or handling of wipes may lead to transfer of germs from one area or surface to another. Additionally, wiped surfaces typically require a finite amount of time for the sanitizing to be effective involving up to a minute or more of vigorous scrubbing not including drying time. Finally, the packaging containing the wipes often is bulky and disposal of used wipes may not be convenient and also adds to landfill waste streams. Therefore use of wipes may not be practical when engaged in common everyday activities while encountering items and/or surfaces that one wishes to quickly sanitize.

There exists, therefore, an unanswered need and opportunity for a portable device that virtually everyone conveniently has with them at all times that also can provide a sanitizing function on a broad class of pathogens wherever and whenever needed.

### BRIEF DESCRIPTION OF THE DRAWINGS

The invention may best be understood by reference to the detailed description taken in conjunction with the accompanying drawings in which:

FIG. 1 illustrates where ultraviolet (UV) light exists within the electromagnetic spectrum and also the relative subcomponents of UV light comprising UVA, UVB, UVC and UVU wavelengths.

FIG. 2 is a representative illustration of inactivation of bacteria as a function of wavelength of light.

FIG. 3A illustrates an accessory case for a portable productivity device, such as a smart phone, in accordance with an embodiment of the invention.

FIG. 3B illustrates an accessory case for a portable productivity device, such as a smart phone, in accordance with another embodiment of the invention.

FIG. 3C illustrates an accessory case for a portable productivity device, such as a smart phone, in accordance with another embodiment of the invention.

FIG. 3D illustrates a power source and a printed circuit control module for an accessory case to receive a portable productivity device, such as a smart phone, in accordance with an embodiment of the invention.

FIG. 3E illustrates a representative UV LED light source for an accessory case for a portable productivity device, such as a smart phone, in accordance with an embodiment of the invention.

FIG. 3F-1 illustrates UV irradiance of an accessory case in accordance with an embodiment of the invention as measured at a distance of 1″.

FIG. 3F-2 is an equivalent table of UV irradiance of an accessory case in accordance with an embodiment of the invention as measured at a distance of 1″.

FIG. 3G-1 illustrates UV irradiance of an accessory case in accordance with an embodiment of the invention as measured at a distance of 1.5″.

FIG. 3G-2 is an equivalent table of UV irradiance of an accessory case in accordance with an embodiment of the invention as measured at a distance of 1.5″.

FIG. 4 illustrates a representative UV light emitting diode (LED).

FIG. 5A illustrates a representative injection molded case bottom portion of an accessory case for a portable productivity device.

FIG. 5B illustrates a representative injection molded case cover portion of an accessory case for a portable productivity device.

FIG. 5C illustrates an accessory case for a portable productivity device as removably assembled from a case bottom portion and a case cover portion and further comprising a UV LED light source.

FIG. 6 illustrates a representative foldable soft accessory case for a portable productivity device.

FIG. 7 illustrates a representative smartphone incorporating an array of sanitizing LEDs in accordance with another embodiment of the invention.

FIG. 8 illustrates an inactivation profile for selected species of bacterium as a function of wavelength.

FIG. 9A is a table of electrical performance specifications for a representative commercially available UV LED driven at 100 mA.

FIG. 9B is a table of electrical performance specifications for a representative commercially available UV LED driven at 40 mA.

FIG. 10A is a table of output power as a function of forward current at constant voltage for a representative commercially available UV LED.

FIG. 10B is a table of relative intensity as function of forward current for a representative commercially available UV LED with LED junction temperature held constant at 25° C.

FIG. 11A is a table of electro-optical characteristics for a representative commercially available UV LED driven at 100 mA.

FIG. 11B is a table of electro-optical characteristics for a representative commercially available UV LED with LED junction temperature held constant at 22° C.

FIGS. 12A and 12B illustrate an accessory case in accordance with the invention being used to sanitize an object.

### DETAILED DESCRIPTION

The following embodiments describe methods and apparatus for making and using a portable sanitizing arrangement comprising an accessory case for a mobile productivity device further comprising a light source for emitting ultraviolet (UV) light for disinfecting objects and surfaces that may be encountered by a user. It will be obvious, however, to one skilled in the art, that the present invention may be practiced without some or all of these specific details. In other instances, well known process operations have not been described in detail in order not to unnecessarily obscure the present invention.

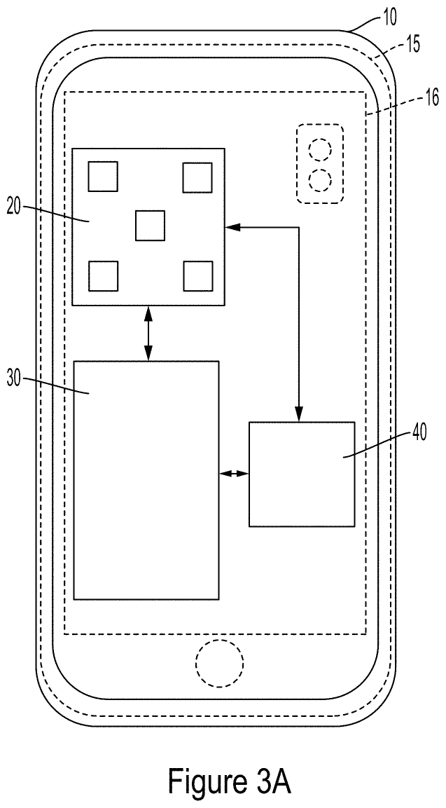

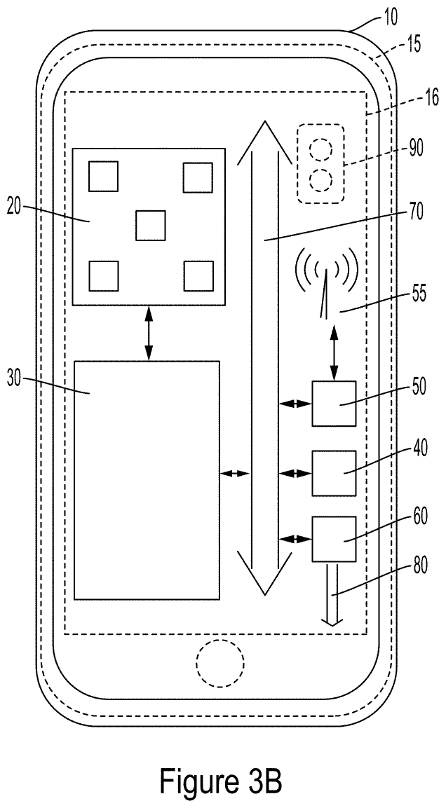

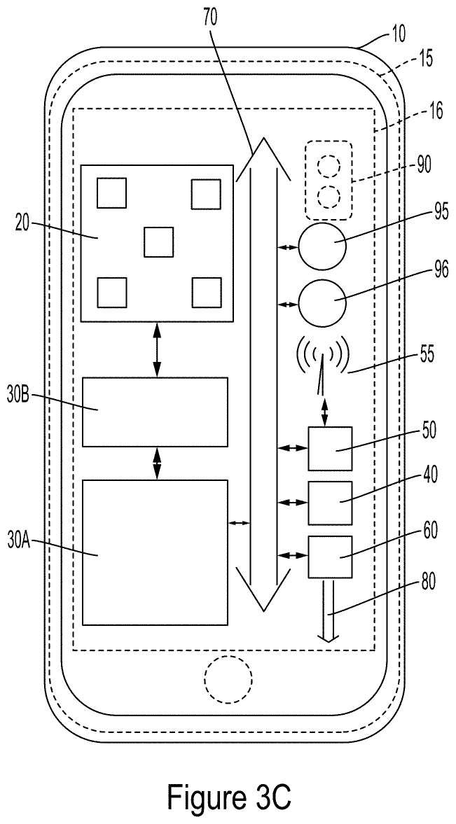

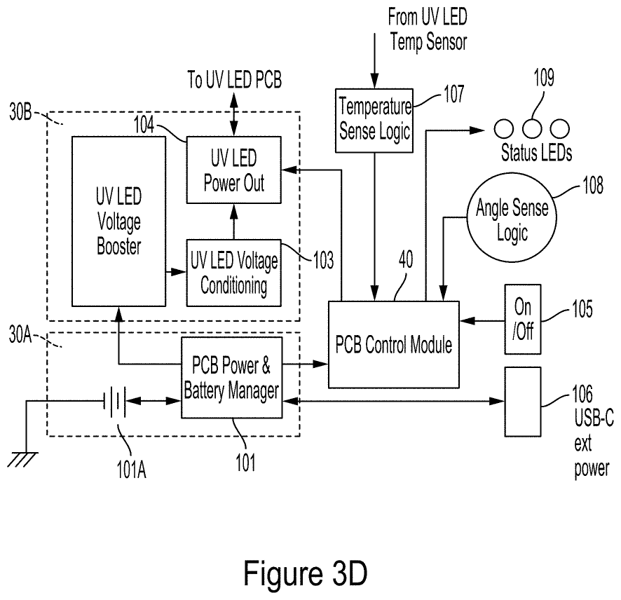

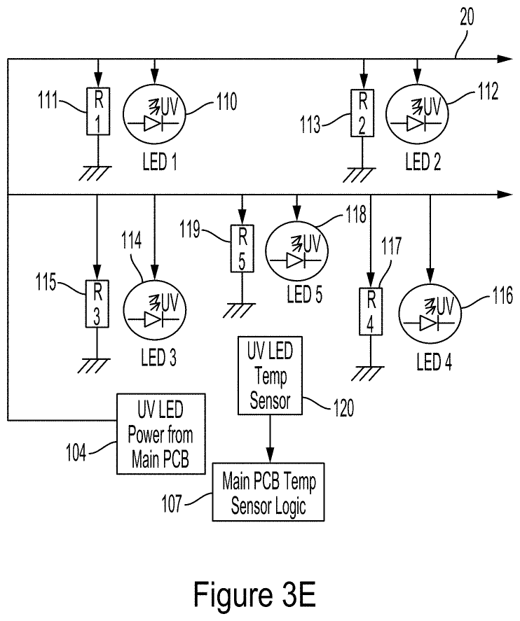

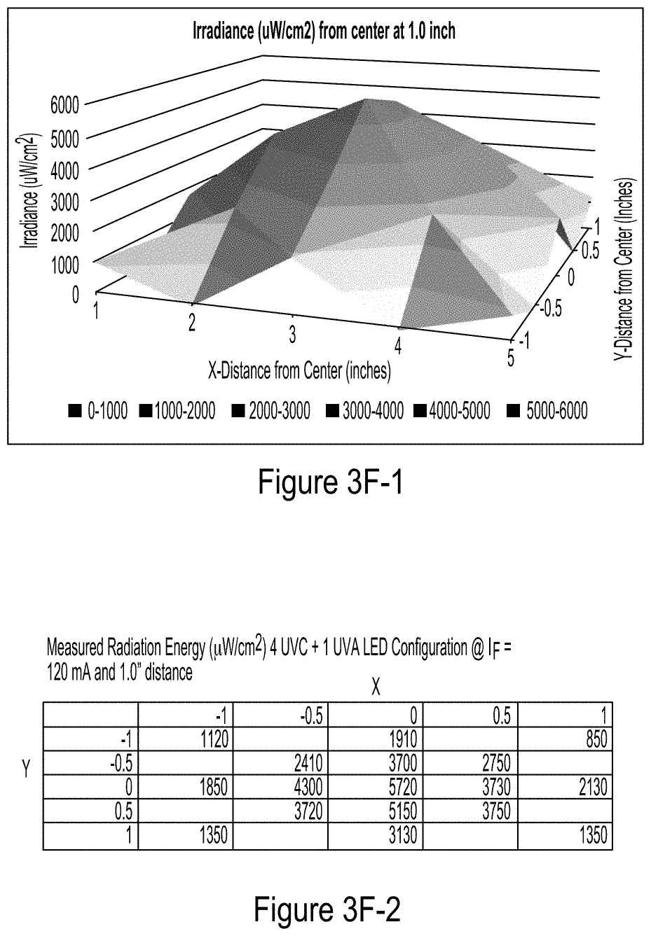

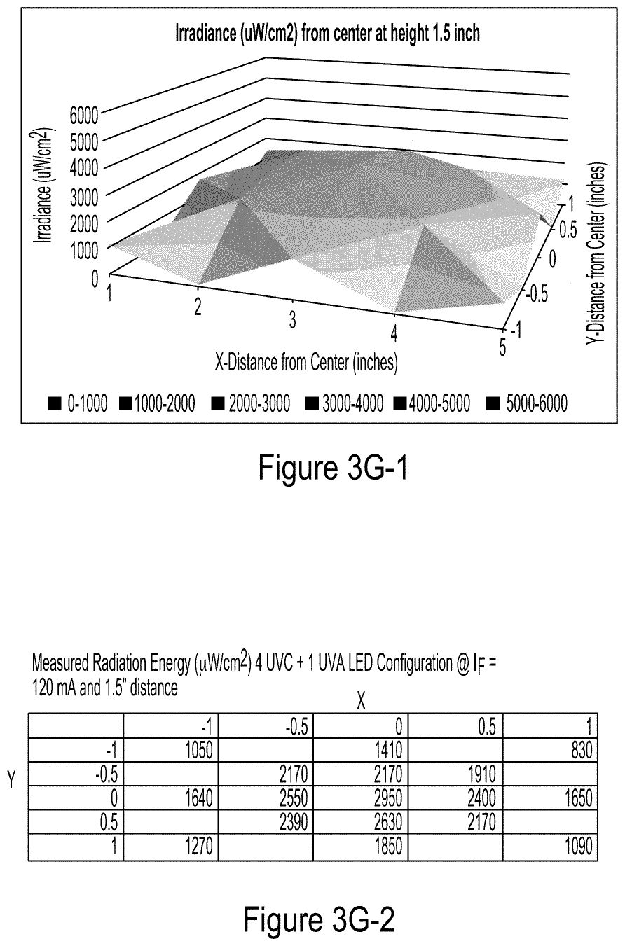

Referring now to FIG. 3A, the sanitizing arrangement comprises an accessory case 10 designed to enclose a mobile productivity device 15, for example a smartphone as shown by a dashed outline. Case 10 further comprises an electrical power source 30 coupled to an ultraviolet (UV) light source 20. The mobile productivity device 15 alternatively may consist of a tablet or a laptop computer among other similar devices. The mobile productivity device 15 is assumed to have a user-facing aspect, or “front side”, comprising primarily a touch-sensitive display screen as suggested by dashed line 16, and also a rear-facing aspect, or “back side”, comprising a structural casing enclosing various functional components of productivity device 15. The accessory case 10 is designed so that productivity device 15 fits snugly within case 10, i.e., with a “press” or other “interference” fit that surrounds and encloses the entire back side of the mobile productivity device 15 and therefore is securely retained within case 10, as suggested by the dashed outline. Necessarily, any user-facing components such as the display screen 16 and other controls of productivity device 15 that must remain exposed to be functional, e.g., camera optics, buttons and switches, must remain uncovered and accessible to a user whenever mobile productivity device 15 is secured within case 10.

With continued reference to FIG. 3A, electrical power source 30 further comprises an electric charge storage device of suitable voltage and current capability together with an energy delivery system that is compatible with the particular mobile productivity device. The charge storage device may comprise a rechargeable lithium cobalt oxide battery, also called a lithium polymer battery, but also may comprise a rechargeable nickel metal hydride (NiMH) cell or a rechargeable nickel cadmium (NiCad) cell. New battery structures and chemistries also are being developed such as grapheme enhanced lithium ion cells that store more charge. Furthermore, the electrical power source 30 may omit a charge storage device altogether and rely only on a constant electric charge generation element such as a miniature fuel cell or even an intermittent charge generation element such as a solar cell. Other types of electrical power source 30 may be feasible depending on the particular shape, form factor and intended functionality of accessory case 10, but for all practical purposes herein power source 30 can be considered to comprise a rechargeable lithium polymer battery, and also as suggested by reference flag 30 in FIGS. 3B and 3C, and further as reference flag 101A in FIG. 3D.

Importantly, the dimensions of the battery portion of electrical power source 30, may influence, that is to say determine, the final shape and dimensions of accessory case 10. In fact, power source 30 may comprise the largest single subsystem within case 10 if it intended for use with a compact productivity device such as a smartphone. More recently, new high capacity yet thin form factor batteries have become available (e.g., Shenzhen Kamcy New Energy Products Co., Ltd. of Shenzhen PRC) and may serve as the charge storage device for power source 30. Battery capacity nominally is rated at 5500 milliampere-hours (mAh) which is substantially equivalent to nominal iPhone battery capacity. The referenced Shenzhen Kamcy battery weighs about 20 grams and is rated to deliver 1A output at 5 VDC. Lateral dimensions of the battery are 40 mm×60 mm with a thickness of just 4 mm, or approximately half as thick as an iPhone. Accordingly it will be appreciated that such a slim form factor battery will not appreciably increase the overall thickness of power source 30, nominally about 4.4-4.5 mm even for a small case 10. For accessory cases being designed for productivity devices other than smartphones, the form factor and volume envelope of power source 30 may not present a significant design issue or limitation.

Electrical power source 30 further may comprise any of several conventional DC power supply and regulation components and features (not shown) that generate sufficient charge flow (current) for the power requirements of the various subsystems of accessory case 10. Such a power supply may comprise a transformer, a rectifier, a filter to remove ripple from the rectifier, and one or more regulator circuits to supply current and voltage at steady, nominal levels. DC power supplies are well known in the art and need not be further elaborated here. Electrical power source 30 further comprises circuitry for recharging the battery, including a physical/wired means such as a USB port, or a wireless power transceiver interoperating with an external charging pad, such as the Qi standard as governed by the Wireless Power Consortium (WPC), or both. Additionally, power source 30 is coupled to manage power, that is voltage and/or current, requirements of all electrical components disposed with accessory case 10 as a whole, either from power source 30 within case 10 or alternatively from a remote power source e.g., as may be embedded within the mobile productivity device 15 disposed within case 10 provided that electrical interconnects are appropriately made.

With brief reference to FIG. 3C, accessory case 10 also may operate with external power applied to case 10 via USB connector 106 or equivalent, for example by means of a conventional external charging device. Electrical charging schemes relying on external devices including wireless charging principles are well known in the art and do not require elaboration here.

In FIG. 3A, case 10 further comprises a control module (CM) 40 coupled between power source 30 and light source 20 and in effect comprising a logical central processing unit of the accessory case 10. For convenience, power source 30 should be understood to supply voltage and current needs for all electrical components disposed within accessory case 10. Therefore the balance of this specification may dispense with express references that power source 30 is coupled to a particular electrical component unless that reference is essential to the teaching and understanding of the component. For simple implementations of the invention, CM 40 may comprise hardware components that do not execute any logical or conditional operations. In theory CM 40 could be realized using only discrete components such as transistors, resistors, op-amps, and flip-flops and the like, but would be unlikely in practice due to limited real estate within accessory case 10 as well as for higher manufacturing cost, power management and reliability reasons.

On the other hand as illustrated in FIG. 3B, in a more complex implementation, case 10 may further comprise hardware and software elements in addition to CM 40 such as a communications management unit (CMU) 50 coupled to a data bus 70 for controlling other functional components within accessory case 10. For such implementations, CM 40 may comprise a semicustom control device such as an application specific integrated circuit (ASIC) or alternatively may comprise an entirely purpose-built processor further comprising at least a central processing unit, a control store/memory for storing executable code, system memory and an I/O interface.

In FIG. 3B, CM 40 further comprises a control executive such as an operating system (OS) rendered in hardware or firmware. The OS manages all machine state operations and data flow within CM 40, as well as processing and responding to commands from the user via an input device such as an on-off switch or outputting data to one or more output devices such as a display screen of a mobile productivity device 15 that is in communication with CM 40 via CMU 50 and data bus 70. CM 40 further handles all housekeeping functions of accessory case 10, including but not limited to at least an operating timer function to track how long light source 20 has been operating as well as at least a reset timer function to prevent accessory case 10 from being turned on if operating time exceeds more than a specified continuous time. As presently preferred, CM 40 will turn on light source 20 for sanitizing operations for a period of 30 seconds (30s) and then turn it off. Thirty seconds was selected to prevent case 10 from overheating due to UV LED thermal output especially if turned on unintentionally, but also because production prototype testing shows that virtually all bacteria present on testing surfaces were sanitized, i.e., inactivated, within a few seconds of application of UVC light at nominal operating power of 3-5 mW/cm². Although as presently configured CM 40 will turn off light source 20 after thirty seconds of operation, a user may elect to further sanitize an object or surface by again pressing the on-off switch. As presently configured, the operational timer function executed in CM 40 in the interests of safety will prevent light source 20 from operating for more than four consecutive sanitation cycles, or approximately two consecutive minutes. After four consecutive sanitation cycles have occurred, CM 40 then will execute a cool down timer function lasting two minutes during which time light source 20 cannot be turned on, thereby in effect imposing a thermal safety feature.

With reference to FIG. 3B and FIG. 3D, in operation CM 40 may receive a user-issued command, such as “turn on” or “turn off” by way of a contact- or capacitively-coupled switch 105. Before executing either command, CM 40 first sends a check status instruction over data bus 70 to poll power source 30 and light source 20 as to its present state of operation. If the present state of operation is “on”, CM 40 will interpret pressing the on-off switch as requesting a power-off operation. If the present state of operation is “off”, then CM 40 will interpret pressing the on-off switch 105 as requesting a power-on operation, respectively. Whenever powering on, CM 40 first performs a system health check to determine if all necessary subsystems such as light source 20, power source 30, and CMU 50 are operating within normal limits as verified by a “system OK” reply signal sent to CM 40 by each subsystem. Thereafter, CM 40 sends a “light source on” command comprising a corresponding “light source on” data packet on data bus 70 to LED power output stage 104 that ultimately connects light source 20 to power source 30 and thereby energizing LED elements comprising light source 20.

Still referring to FIG. 3B, CMU 50 (coupled to the CM 40 via data bus 70) serves as a data traffic manager for all communications between CM 40 and all internal components of accessory case 10 as well as any peripheral components connected to accessory case 10. CMU 50 and data bus 70 may operate under a conventional or customized control protocol and language adapted for peripheral devices. Known examples of peripheral control protocols are RS-232, SCSI, SCSI2 among many others as circumstances may require.

Additionally, wireless communications between accessory case 10 and portable productivity device 15 and/or other devices may be enabled by embedding the well-known Bluetooth protocol in both case 10 and productivity device 15 by way of Bluetooth-compliant components, thereby enabling communication extending up to several meters. Bluetooth is a well-known low power wireless communications protocol originally developed at Ericsson Communications with the collaboration of IBM and standardized under IEEE 802.15.1 but now is managed by the international Bluetooth special interest group (SIG).

Another widely accepted and implemented wireless communications protocol is standardized under IEEE 802.11, commonly referred to as “WiFi”. In contrast to the Bluetooth IEEE 802.15 standard which is basically a point-to-point protocol, the IEEE 802.11 wireless protocol can support many devices simultaneously including networked devices operating over a much greater distance.

Alternatively or in combination with the foregoing standards, CMU 50 may implement a near-field active and passive communications protocol also known as NFC and standardized under ISO 18092. NFC typically extends over a much short path length of a few centimeters. Interconnection between the CMU 50, an antenna 55, and any wirelessly controlled peripherals necessarily would require compliance with radio and data communications implementations such as physical and transport layers as known in the art.

With continued reference to FIG. 3B, IOC 60 is coupled via data bus 70 to CM 40 and CMU 50. IOC 60 controls all signal and control data traffic into and out of accessory case 10 via an I/O bus 80. I/O bus 80 may operate under a conventional serial communications protocol such as Intel's eSPI communications standard and available as a free license from Intel. Between the two, CM 40 and IOC 60 manage all data traffic, both within case 10 and also between case 10, the mobile productivity device 15 that is disposed within case 10, as well as other external peripheral devices that may be in communication with case 10 besides the productivity device, for example an external display or another mobile device. Communications between the referenced components and devices may relate to user-issued commands, machine status inquiries, safety interrupts, device initialization, image capture and transmission, LAN and WAN network and cloud connectivity, among many others.

Still with reference to FIG. 3B, UV light source 20 comprises at least one solid state light emitting diode (LED) device disposed in the accessory case and producing light in the UVC wavelength range of about 200-280 nm, and in one embodiment is centered about 275 nm±5 nm. In operation, each LED device is anticipated to produce an optical output power of several milliwatts, typically ranging from about 2-8 mW at a nominal forward current of about 100 mA. In theory, light source 20 could comprise other sources of UVC light such as discharge lamps. However in practice such lamps would be too bulky and consume too much power for the present application that prioritizes compactness and portability. Wavelength ranges that are effective for inactivating bacteria, viruses and other pathogens are discussed in more detail below.

The LED device(s) comprising UV light source 20 have a protective lens and are disposed laterally within an anterior external surface of accessory case 10 and at such a depth within case 10 so that the protective lenses of the LEDs essentially are coplanar with the anterior external surface of accessory case 10, or as the manufacturer otherwise may best determine. The number and spacing of LED devices forming light source 20 may vary by size, power consumption, dispersion angle and output wavelength range so that a user may select from different styles and prices according to how much power, irradiance, wavelength and surface area coverage that a user may require.

Importantly, one embodiment of case 10 incorporates a plurality of LEDs operating at a plurality of wavelengths to achieve a more broadband sanitizing performance, or alternatively a wavelength spectrum with selected UV spectral peaks for optimal disinfection depending on the pathogen in question. Notably, such spectral peaks may comprise UVC and other wavelengths including far UVC, UVA, and near visible UV. Additionally, the benefits of this invention also may be obtained with LEDs centered about a single wavelength, with bandwidths varying from narrow to broad as may be needed for a particular application.

Accessory case 10 further may comprise electrical conductors and connections of such quantity and type to enable LED devices comprising light source 20 to be functionally arranged across the anterior external surface in various arrangements to achieve a desired dispersion pattern at an optimal operating distance from a target surface or object.

Importantly, multiple LEDs may be used to enable accessory case 10 to produce a convolution of multiple wavelengths and irradiance intensities of UV light, the precise number of LEDs being selected to achieve sanitation conditions of desired dosage and wavelength as may be needed to inactivate targeted species of pathogens. As previously noted, individually packaged UVC LED devices, emitting wavelengths in a range of about 250-280 nm, are now commercially available, and shorter wavelength LEDs are now in laboratory prototype stage. Typical dimensions of such LED devices are about 4 mm×4 mm×1.3 mm (L, W, H). A single LED device of the foregoing dimensions, such as advertised by International Light, has a radiant flux of about 3-18 mW at a forward voltage of about 5-8 volts. When embedded into an array of multiple LEDs comprising light source 20 is sufficient to achieve sanitizing/disinfecting dosage within reasonable time spans, in most cases a few (1-5) seconds of dwell time over a given surface area of defined dimension.

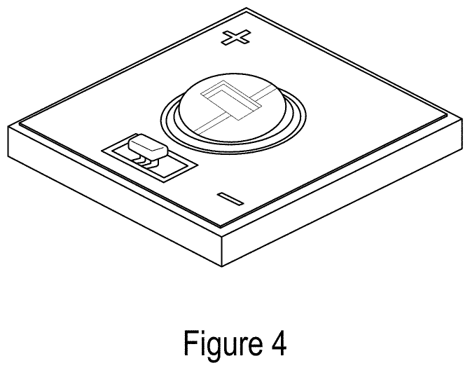

With brief reference to FIG. 4, Bolb Inc. of Livermore, Calif., USA, offers LED chips (as shown in FIG. 4) that reportedly delivers about 4-9 mW of UVC power at wavelengths ranging from about 265-275 nm from a single 0.5 mm×0.25 mm LED when operated at 20 mA forward current and about 5-7 V forward voltage. Bolb also offers pre-packaged strips of LEDs for applications requiring greater fluence.

Referring now to FIG. 3C, another embodiment of the invention is shown comprising a power booster 30B for amplifying the voltage supplied by electrical power source 30A. Some design selections for electrical power source 30A may not require a power booster, depending on the voltage and current characteristics of the power source 30A that is selected as well as the forward current and voltage necessary to operate the UV LEDs. Although the embodiment shown in FIG. 3B functions as shown and described herein, the embodiment shown in FIG. 3C includes power booster 30B for driving the UV LEDs comprising UV light source 20 when the nominal voltage of the battery component of electrical power source 30A selected in this embodiment is lower than the minimum voltage necessary to turn on UV LEDs comprising light source 20. For example, UV LEDs made and sold by Semicon of South Korea require a forward voltage of approximately 7.5 V, which is greater than the battery (drawing element 101A in FIG. 3D) can deliver. Note that even if power source 30A voltage output were sufficient to drive the UV LEDs without further amplification, a power booster 30B could be used to amplify the voltage delivered to light source 20 even higher and thereby drive light source 20 to even greater output. In so doing, the time needed for any given disinfecting cycle may be reduced.

As illustrated in both FIGS. 3B and 3C, accessory case 10 further may comprise at least one image recording element 90 further comprising a lens and an image capture device such as a camera. Image recording element 90 further may comprise one or more optical filter elements (not shown) disposed between the lens and the image capture device 90 for the purpose of enhancing visibility of contaminant species existing on an object or surface, for example by means of interference fringes, dark field illumination, and/or fluorescence. The accessory case 10 may be coupled to the mobile productivity device by way of physical interconnect lines 80 or alternatively by a wireless communications protocol such as Bluetooth. The purpose and function of lens and an image capture device 90 is described in further detail below.

With reference now to FIG. 3D, a more detailed illustration is shown of selected internal components of the invention shown in FIG. 3C. In FIG. 3D, electrical power source 30A, power booster 30B, command module 40 as well as temperature sensor 107 and angle sensor 108 are coupled to mutually communicate via data bus 70 shown in prior FIGS. 3B and 3C. In the embodiment shown in FIG. 3D, power source 30A further comprises a printed circuit board (PCB) power and battery manager 101 coupled between a USB-C power port 106 and an on-board battery 101A. PCB power and battery manager 101 within power source 30A in practice manages the power requirements of accessory case including supply power to other case components as well as the recharging of battery 101A from an external power source, e.g., a recharging accessory or external battery (not shown). Status LEDs 109 are coupled to power source 30 as well as CM 40 to provide a user with status information as to operational condition of battery 101A. As implemented, status LEDs comprise green, red and blue indicators that, when illuminated, respectively indicate conditions wherein the battery is fully charged, is charging, or requires charging. Additionally, the blue status LED flashes whenever an over-temperature condition exists either in the main PCB or in UV light source 20. Obviously, other combinations of status indicators, colors and conditions are possible.

As is also shown in FIG. 3D, power booster 30B further comprises a voltage booster 102, a voltage conditioner 103, and a power output stage 104 that is coupled to light source 20. In FIG. 3C, voltage booster 30B may comprise, for example, a voltage to frequency converter implemented as an LC equivalent circuit and then increasing the frequency before converting back to an amplified DC voltage signal at a specified reference voltage established by a Zener diode, e.g., by means of a rectifying diode as known in the art. Voltage booster 102, voltage conditioner 103 and LED power output stage 104 in concert serve to deliver voltage and current sufficient to reliably operate light source 20 as desired; however, the specifics of these components are variable to the degree that one skilled in the art may design them to achieve other, that is to say different, operational objectives.

In FIG. 3D, control module 40 in addition to managing the overall operation of accessory case 10, also is coupled to receive various inputs from one or more on-board sensors for purposes of product safety. In FIG. 3D, angle sensor 108 monitors the attitude, i.e., angular displacement from horizontal, of accessory case 10 when in use. Because accessory case 10 is designed and intended to be operated in an essentially horizontal position, angle sensor 108 will issue an excess angle signal to CM 40 if case 10 is held at an angle greater than about 60° from horizontal, irrespective of azimuthal orientation. The intent/purpose behind angle sensor 108 is to prevent operation of light source 20 if case 10 is excessively angled upwards such that light emanating from light source 20 inadvertently could shine on the user or an adjacent person, or if case 10 were to be inverted. Although is it believed and indeed has been reported that far UVC light likely is not harmful to human skin and/or eye tissue, angle sensor 108 is embedded within the case 10 as an added measure of operational safety until all such concerns are resolved.

Similarly, case 10 also comprises a temperature sensor 107 to detect whether case 10 when operating exists in an over-temperature condition due to evolution of heat within case 10 and more specifically from light source 20. When operating, the UV LEDs comprising light source 20 each consume several milliwatts of power, some of which is converted to UV light but most of which is transformed into heat and therefore may become quite warm during use. Thermal management is accomplished through a combination of conductive and insulating materials as well as heat sinks mounted to all heat producing components within case 10 as deemed necessary.

Reference now is made to FIG. 3E illustrating an embodiment of light source 20 disposed within accessory case 10. In FIG. 3E, a plurality of UV LEDs 110, 112, 114, 116 and 118 is shown (LEDs 1-5) coupled to UV LED power 104 coming from a main PCB. As shown, all LEDs 1-5 are coupled in parallel with each UV LED device having its own voltage adjustment means 111, 113, 114, 117 and 119 respectively (e.g., resistors R1-R5), with each resistor R1-R5 serving in effect as a voltage divider to deliver appropriate voltage to LEDs 110, 112, 114, 116 and 118 as may be required. The parallel arrangement enables light source 20 to comprise any desired number of LEDs subject only to the voltage and current limits of power source 30. As is further shown in FIG. 3E and for the same reason as stated above, light source 20 also may comprise its own temperature sensor 120 coupled to temperature sensor 107 (FIG. 3D) that will issue an over-temperature signal to CM 40 if light source 20 exceeds a pre-established over-temperature condition. In such a case, i.e., if the operating temperature of light source 20 exceeds a programmed/pre-established over-temperature condition, in the present case approximately 50° C., then CM 40 according to its programmed logic will issue a “light off” command to UV power output stage 104 and thereby de-energize light source 20 within accessory case 10. It should be noted that all LEDs comprising light source 20 are operated at a duty cycle such that the LEDs appear “on” to the human eye, accounting for persistence of vision.

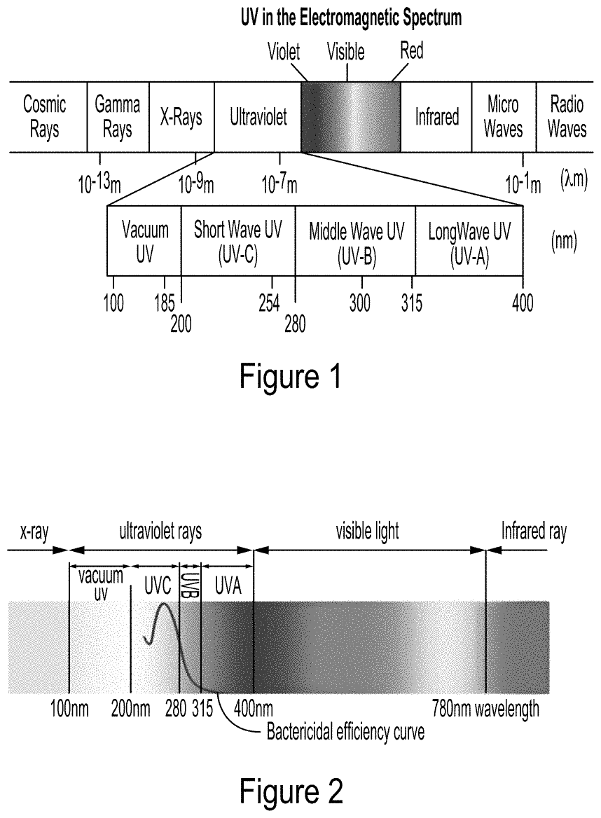

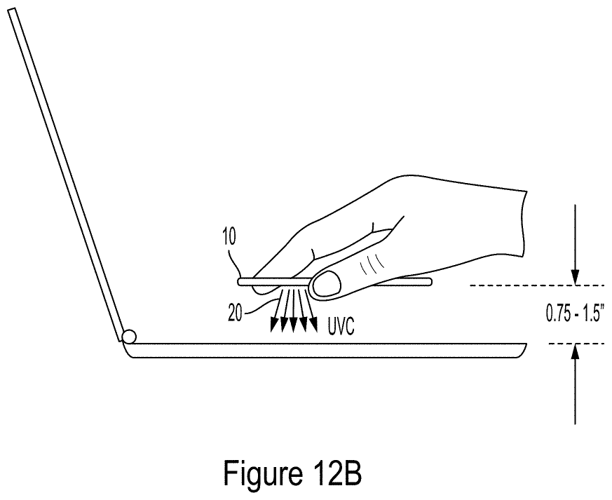

Referring now to FIG. 3F-1 and FIG. 3F-2, illustrated are irradiance (i.e., energy) plots and data respectively for UV light produced by four UVC LEDs and one UVA LED comprising light source 20 as measured 1″ below the centrum of light source 20 and along x- and y-directions emanating laterally from the centrum of light source 20. In FIG. 3G-1 and FIG. 3F-2, the combined power of four UVC LEDs plus one UVA LED comprising light source 20 is shown in units of μW/cm², but which also may be conveniently rescaled to mW/cm² or mJ/sec-cm² by dividing by a factor of 10³, where 1 W=1 J/sec. As can be seen, the irradiance is greatest near the centrum and diminishes as distance from the centrum increases. However, as shown, the irradiance generally remains above 1 mW/cm², which generally is sufficient to sanitize many species of pathogens within an exposure area of about 1″ (2.5 cm) from the centrum in any direction. Therefore, the irradiance plots shown in FIG. 3F-1 and FIG. 3F-2 suggest that case 10 produces an effective sanitizing area of about 2″×2″=4 in², or approximately 10 cm² when case 10 is held at a distance of about 1″ (2.5 cm) from a surface or object to be sanitized. For surfaces and/or objects greater than about 4 in², the recommended method of using accessory case 10 entails moving the case in a slow horizontal scanning fashion such that a requisite dose needed to sanitize, that is disinfect, the entire area is achieved, as will be further described below in connection with FIGS. 12A-12B.

Similarly, in FIG. 3G-1 and FIG. 3G-2, illustrated are irradiance (i.e., energy) plots and data respectively for UV light produced by the same four UVC LEDs and one UVA LED comprising light source 20 but this time measured at a distance of 1.5″ below the centrum of light source 20 and along x- and y-directions emanating laterally from the centrum of light source 20. In FIG. 3G-1 and FIG. 3G-2, the combined output power of four UVC LEDs plus one UVA LED comprising light source 20 remains the same but as can be seen, measured irradiance is relatively diminished compared to that shown in FIG. 3G-1 and FIG. 3G-2 due to the greater distance from the centrum. Even so, the irradiance generally still remains above 1 mW/cm² which is reportedly sufficient to achieve sanitizing, that is disinfection, for many species of pathogens over the same sanitizing area of about 2″×2″ or 4 in² (˜10 cm²) described above. For surfaces and/or objects greater than about 4 in², the method of using accessory case 10 at greater distances again entails moving case 10 in a slow, horizontal scanning fashion provided however that a requisite dose needed to sanitize, i.e., disinfect, the entire area may require more exposure time depending on the species of pathogen. This distance vs exposure effect will be further described below.

As suggested previously, accessory case 10 as variously shown in FIGS. 3A-3E may comprise LEDs that individually and/or collectively emit a plurality of UV wavelengths (or ranges of UV wavelengths), as may be dictated by commercial availability, pricing and efficacy of UV LEDs and also total dose (as defined below) required to sanitize pathogens of interest. For example, additional wavelengths closer to the visible spectrum, such as UVA at around 395 nm, may be achieved by adding one or more UVA LEDs within the array of UV LEDs disposed in case 10 because it is theorized that some mixing, i.e., constructive and destructive interference, of convolved UVA and UVC wavelengths enhance sanitation efficacy for some microorganisms and thereby reduce the time needed to achieve a given amount of sanitation. Analogously, similar sanitation efficacy benefits may be derived from mixing UVC wavelengths, say 250-280 nm, with far UVC wavelengths, say below about 230 nm.

Example Device Specifications and Operational Conditions

The following device specifications and operational conditions may be used to realize aspects of accessory case 10. It should be understood that these are only example specification of one implementation, and other configurations may be possible consistent with alternative constructions that are within the spirit and scope of the claims. With this in mind, example specifications include but are not limited to:

1. Battery type capacity and output: rechargeable Li-ion 1500 mAh @ 3.7V

2. All LEDs driven at specified voltages by adding resistors in parallel

3. UVC LEDs: 270-280 nm Semicon, 15-17 mW flux @ 120 mA, 6.5V

4. UVA LEDs: 390-400 nm, 60-80 mW flux @ 60 mA, 3.2V

5. Power on/off button aligned to USB-C port right edge

6. USB-C port for charger cable

7. UVC LEDs connected in parallel

8. UVA LED power on at the same time with UVC LEDs, max delay+/−1 sec

9. No flickering during 60 sec for both UVA and UVC LEDs

10. 30 sec “Time sensor” shut-off function

11. 360° angle protection sensor (OFF: Face Up, ON: Face Down)

12. Charger circuit with LED status indicator(s) as follows:
green=full charge
red=charging
blue=flashes when battery low or over-temperature)

13. Battery connection: 51005 2P

14. Max temp of LED heat sink=100° C. after 5 min operation

15. Max temp of PCB module=50° C. after 5 min operation

16. Over-temperature protection sensor for LED board at 65° C.

Example Case Construction Details

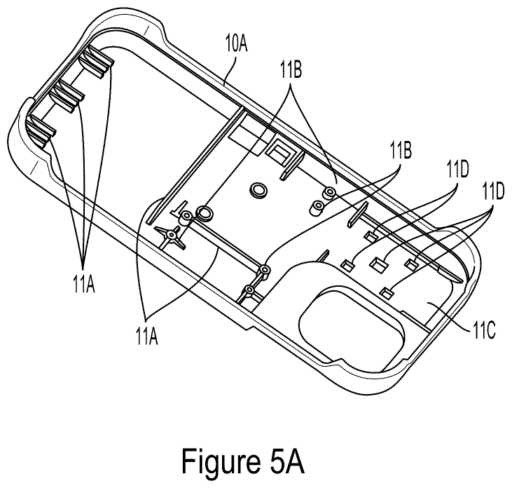

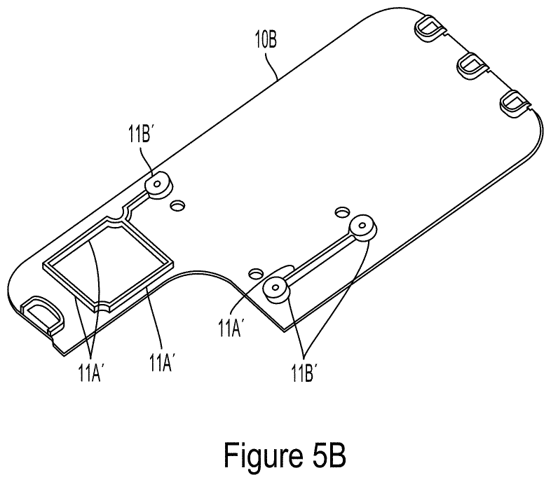

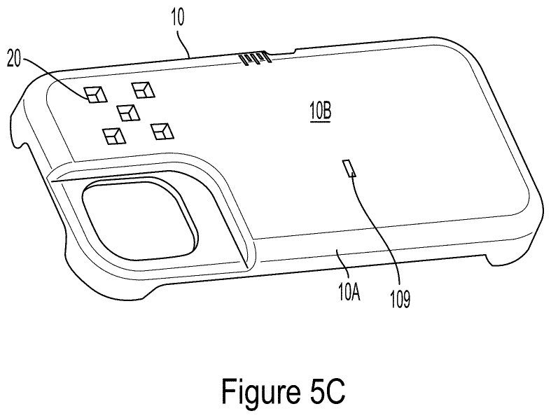

It is anticipated and assumed that accessory case 10 referenced in FIGS. 3A, 3B and 3C will be sized and shaped to accept, i.e. receive, any of numerous styles and shapes of portable productivity devices. That is, case 10 can be designed to fit specific models of cell phones, tablets or other so-called personal digital assistants (PDAs), as well as laptop computers as may be desired. Size, shape and material used to form accessary case 10 are anticipated to vary by manufacturer and may include but are not limited to, rigid thermoplastics such as polycarbonates, various powders and polymer aggregates suitable for additive manufacturing processes such as three-dimensional printing, and/or injection moldable plastics such as thermoplastic elastomers and liquid silicone rubbers. Referring now to FIG. 5A, shown is a three-dimensional rendered image of an injection-molded case bottom portion 10A partially comprising case 10. In FIG. 5A, the case bottom portion 10A typically will feature molded-in strengthening ribs 11A and bosses 11B as shown to respectively enhance rigidity of case bottom portion 10A and also to facilitate assembly/disassembly of case 10 with a plurality of fasteners (not shown) as may be appropriate for a given design. Case bottom portion 10A also comprises a recess 11C to receive a UV light source printed circuit assembly (not shown) along with a plurality of through-holes 11D to permit UV LEDs mounted on the UV light source printed circuit assembly to emit their radiation outward through corresponding openings in case 10. Similarly, shown in FIG. 5B is a complementary injection-molded case cover portion 10B partially comprising case 10 that is designed to mate with case bottom portion 10A and be removably secured thereto by a plurality of fasteners (not shown). Case cover portion 10B similarly comprises strengthening ribs 11A′ and bosses 11B′ to respectively enhance structural strength and to facility assembly and disassembly of case 10. When case cover portion 10B is mated to case bottom portion 10A, it forms an included internal volume between case bottom portion 10A and case cover portion 10B thereby protecting all functional electronic components with case 10 from physical and environmental harm.

With brief reference to FIG. 5C, shown is an assembled case 10 after case bottom portion 10A and case cover portion 10B have been joined together and secured by suitable fasteners (not shown). Functionally, the LEDs comprising light source 20 face outward through openings formed in case bottom portion 10A, as do status LED(s) 109 through a corresponding opening formed in case bottom portion 10A. As noted above case 10 can be disassembled when needed by slackening and removing such fasteners.

A skilled person will understand that compliant natural and synthetic materials such as leather, vinyl and the like may also be used to form the accessory case provided that first, all active electronic components are securely housed/disposed within an interior volume of case, and secondly, that appropriate friction elements are included to provide sufficient “grip” on whatever productivity device is retained within accessory case 10 under ordinary use conditions Additionally, accessory case 10 may be designed and manufactured to accommodate larger models of productivity devices, including flat, tablet-like designs as well as folding devices having a hinged or flexible cover such as a laptop computer or a tablet device with a detachable/foldable keyboard. Beyond the foregoing however, accessory case 10 also may embody several additional devices, features and functions as described below.

### Additional Embodiments

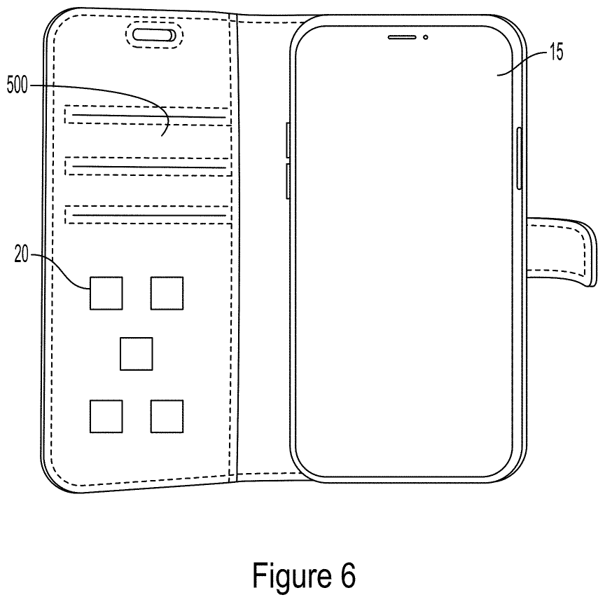

With brief reference to FIG. 6, another embodiment of the invention may comprise a compliant folding case that, in addition to surrounding and cushioning the mobile productivity device, also comprises a foldable cover 500 to help protect the front side of an enclosed productivity device It is anticipated that foldable cover 500 at times may be opened, i.e., moved away from the touchscreen surface of the device when in use, and at other times may be closed, i.e., moved towards or immediately adjacent, the touchscreen surface of the device when stowed or not in use. It should be noted that the folding case shown in FIG. 6 necessarily comprises all components and functions of case 10 described above, including UV light source 20. Moreover, it will be understood that the folding case shown in FIG. 6 may vary dimensionally to accommodate various different power sources, light sources, and other interconnect and functional components as may be desired to fit within such a folding case. Alternatively any UV light source disposed within the folding case may be powered by the power source integrated into the productivity device and coupled to the folding case, either physically via interconnect lines or wirelessly, such as via Bluetooth protocol as described above. Additionally a skilled person will understand that the light source disposed in the folding case further may comprise one LED device or an array of LED devices of such size, power and lateral arrangement as may be optimally determined by the folding case manufacturer for a given specified performance as with rigid case designs described above.

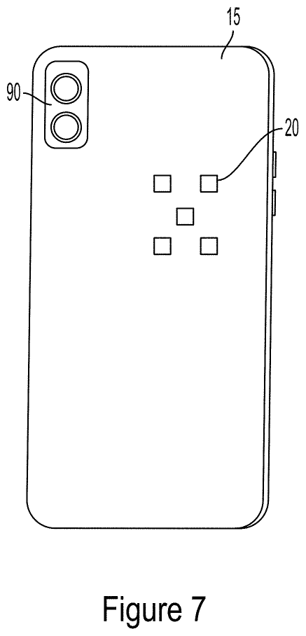

Referring now to FIG. 7, an alternative embodiment of the invention is shown in which a UV light source 200 may be embodied directly within a mobile communications or productivity device, thereby obviating the need for a separate case 10. The productivity device typically may comprise a smartphone such as an iPhone model made and sold by Apple Inc. or a Galaxy model made and sold by Samsung Electronics. However, it is anticipated that many if not most users of such devices will naturally choose to protect their communications devices with an external protective case, including but not limited to the sanitizing accessory case 10 of the described invention. Accordingly, it will be appreciated that some consumers may elect to purchase smartphones or other mobile productivity devices with integrated UV light source 200, whereas others may elect to buy an accessory case such as described herein for use with a productivity device without a UV light source.

In each instance, the productivity device shown in FIG. 7 comprising integrated UV light source 200 that projects UV light comprising a wavelength (or a range of wavelengths as the case may be) that is effective in neutralizing and/or destroying microorganisms and/or pathogens existing on external surfaces of tangible objects, preferably in the UVC range of 200-280 nm. However, UVC wavelength distribution and range may vary about a center wavelength of about 275 nm for practical considerations, for example, such as ample commercial availability and competitive pricing of UVC LEDs operating at this wavelength. However, additional LEDs having different wavelengths may be chosen closer to the visible spectrum, such as UVA at around 395 nm by adding one or more UVA LEDs within the array disposed in the productivity device. UVA wavelengths are added to UVC wavelengths because it is theorized that some mixing, that is the constructive and destructive interference of convolved UV wavelengths may enhance sanitation efficacy with respect to some microorganisms and thereby reduce the time needed to achieve a given amount of sanitation. It is also theorized that different wavelengths may inflict different destruction mechanisms on a given pathogen. For example, 260-280 nm may attack nucleic acid of the microbe whereas shorter wavelengths, say <230 nm, may play a more active role in destroying outer protein layers of the microbe.

With continued reference to FIG. 7 but bearing in mind the arrangements shown in FIGS. 3A-3E and also FIGS. 5A-5C UV light source 200 may comprise one or more UV LED elements in various spatial arrangements disposed within a back (i.e., posterior) side of the mobile productivity device itself, e.g., a smartphone. When integrated within a mobile productivity device as shown in FIG. 7, UV source 200 would be powered by whatever internal, onboard power source that powers the productivity device. Regardless of specific LED element placement, or the number and wavelengths of UV light, it is anticipated that light source 200 will operate at specified optical wavelengths and irradiances optimized to neutralize and/or destroy pathogens of interest, typically ranging from about 200-280 nm and 1-5 mW/cm² respectively.

As suggested by the productivity device shown in FIG. 7 with integrated UV light source, a UV light source alternatively may be embedded in a back surface of a tablet mobile productivity device. Practically speaking there is not much difference between a smartphone and a tablet other than dimension and weight. Further still, it will be appreciated that such a UV light source also may be embedded in a laptop computer cover (not shown), for example in a bezel surrounding a display screen. In the latter case, the UV light source could be caused to irradiate and thereby sanitize a keyboard disposed within the laptop computer whenever the cover is in the closed position. Additionally, such a UV light source in the surrounding bezel of a laptop computer when held in a manner to project UV light away from the user, may be used to irradiate proximate surfaces and objects of interest encountered during the user's day, including but not limited to desk surfaces, desk telephone and computer keypads, door knobs, public point of sale devices, elevator buttons and so on. However use of a laptop configured in such a manner admittedly would be far less convenient in comparison with a smartphone having similar UV light sources as illustrated in FIG. 6 or a tablet device.

Referring now again to FIG. 3B and FIG. 3C, accessory case 10 may comprise an image sensor 90 to capture and identify organic contamination that may be present on surfaces before the UV light source is engaged, for example by inducing fluorescence. Fluorescence is a well-known phenomenon of some materials that absorb light of one wavelength and then emit light at another wavelength that is characteristic of that material. Fluorescence may be associated with inorganic materials, but more often is associated with organic or biological materials that absorb UV light and in turn emit visible light. Therefore fluorescence can be used to identify various organic species, including germs. Light source 20 within case 10 or other LEDs integrated within accessory case 10 or alternatively with the productivity device that radiate at specific frequencies known to induce fluorescence in target contaminants may enable identification of certain species that may not be visible to the naked eye. With brief reference again to FIG. 3B and FIG. 3C, image sensor 90 in conjunction with a display screen of the productivity device disposed within case 10 thereby functionally would comprise a fluorescence imaging device (FID). In operation, the FID would record such fluorescent radiation, filter out non-fluorescent or destructive wavelengths and then store a digital video image in a buffer within case 10 or alternatively in the on-board memory of the productivity device. The stored digital video image of any such fluorescent biological material may be then transmitted from the FID via the I/O bus 80 to the CM 40 for further storage, processing, transmission and ultimately display on an image display device, for example the display screen of the productivity device housed within the accessory case 10. It should be noted that image sensor 90 optionally may comprise a plurality of optical filter elements that may be selectively illuminate different bands of fluorescent light and thereby highlight different species of biological materials depending on each material's particular fluorescence-emitting characteristics. Image sensor 90 further may comprise optical or electronic magnification for improved viewing fluorescent objects and materials on the display screen. In some cases, image sensor and optical filters 90 and the FID may serve to magnify the subject so as to present a larger image to the eye in the manner of a microscope.

Another embodiment of the invention may comprise means for visibly highlighting a nominal target area that is within effective sanitizing range for light source 20 at its nominal operating distance. In such an embodiment, case 10 would further comprise at least a visible light projection source, for example one or more broadband white LEDs, coupled to power source 30, command module 40, and communications management unit 50 via data bus 70. In this embodiment, the visible light projection source further would include a shroud or stencil that would shield or “cut off” light beyond a specified boundary corresponding to the effective sanitizing area. When light source 20 is turned on and operating, CM 40 then also issues a “visible light on” command to the visible light projection source to energize the visible illumination source within visible light projection assembly 150 and thereby causing a visible light pattern to be projected from case 10 onto the surface or object that, when in focus at the optimal operating distance of 0.75-1.5″ (1.9-3.8 cm), essentially “paints” a representation approximately equivalent to the lateral boundaries of the known effective sanitizing area. Thus a user of case 10 would be aided by a visible representation of the effective cleaning area that is covered with each successive oscillatory sweep of case 10, similar to how a “gobo” lighting fixture paints a design, logo or wording on a wall or onto a floor.

With specific reference to FIG. 3C, yet another embodiment of accessory case 10 comprises a distance-sensing module 95 to aid in positioning case 10 at an optimal separation distance from a surface or object to be sanitized. Distance sensing module 95 may comprise, for example, a capacitive proximity sensor, a transmitter unit (not shown), a receiver unit (not shown) and a near field antenna (not shown and different from antenna 55). The transmitter unit, the receiver unit and the near field antenna together comprise an end-to-end wireless communications system that will be connected together when contacts within the distance-sensing module 95 are “closed” whenever a surface or an object is detected. In this embodiment the distance sensing module 95 also comprises a sensing aperture mounted against the rear-facing or “back” surface of case 10 such that the sensing aperture of sensor 95 faces away from the front aspect of the case 10 and toward whatever surface or object is to be sanitized. The transmitter unit and the receiver unit are disposed within an interior volume of case 10 and coupled to the power source 30A-30B, along with the distance-sensing module 95. In this embodiment, when case 10 along with the enclosed smartphone are held above and parallel to a surface or object to be sanitized and the user presses the “on” switch within case 10, CM 40 would transmit a “power on” command to the power source 30A-30B, to supply regulated DC power to the proximity sensor 95, the transmitter unit and the receiver unit. Thereafter, case 10 is moved laterally as described below and substantially within a plane defined by the optimal separation distance between case 10 and the surface or object to be sanitized, approximately 0.75-1.5 inches (1.9-3.8 cm). So long as case 10 remains within the optimal separation distance, proximity sensor within distance-sensing module 95 will remain “closed” and both the transmitter and receiver units will remain in communication. Evidence of the “closed” condition can be outwardly indicated by a user-visible indicator, such as an LED emitting visible light of a chosen color, say green. However, if case 10 (and by implication the productivity device disposed therein) exceed either the lower bound or upper bound of the optimal separation distance between case 10 and the surface or object over which case 10 together with the smartphone are being moved, the contacts within proximity sensor of distance-sensing module 95 will open and thereby signal the “out of band” condition and break off the communications connection between the transmitter unit and the receiver unit. When the transmitter and receiver units no longer are in communication, then CM 40 will cause the visual indicator to change to a complementary or opposite color signaling to a user that case 10 is out of range from the surface and either should be brought closer to the surface or moved farther away, as the case may be to restore the optimal 0.75-1.5 inches (1.9-3.8 cm) separation distance between case 10 and the surface or object to be sanitized. Alternatively case 10 may comprise an audio emitting device such as a speaker that would issue an auditory alert sound, such as a beep or a chirp, whenever case 10 is out of range from the surface and either must be brought closer to the surface or moved farther away. It is anticipated and expected that a user will personalize the auditory alert sound to be personalized or uniquely different from any other system auditory alert such as incoming email or text. It should be noted that this embodiment is not limited to capacitive proximity sensors. Other types of distance measuring sensors may be adapted to measure the optimal separation distance, including but not limited to infrared sensors, ultrasonic sensors, optical sensors and the like. However, each type of sensor must be carefully evaluated for suitability and then optimized for the relatively small separation distances at which this embodiment is expected to function.

Yet another embodiment of accessory case 10 assumes and depends on interoperation of components and subsystems within both accessory case 10 and a mobile productivity device 15 disposed therein, for example a smartphone. In contrast to the immediately forgoing embodiment, this embodiment necessarily requires close cooperation between accessory case 10 and the mobile productivity device disposed therein, and would be enabled by one of two alternative means, namely either by means of CM 40 or other control module within case 10 executing an instance of the mobile device operating system that is running on the mobile productivity device and thereby communicating directly with and controlling the productivity device, or alternatively by means of a utility application (frequently referred to now as an “app”) running on the mobile productivity devices that manages and authorizes the interaction between case 10 and the mobile productivity device. Accordingly in the case of an Apple iPhone running iOS, case 10 also would need to run the same or a compatible version of iOS. Analogously, in the case of an Android phone such as a Samsung Galaxy, case 10 would need to run the same or a compatible version of the Android operating system running on the Galaxy phone. In effect, this embodiment relies on the ability of case 10 to “mirror” the operating system and processes running on the target mobility device. It should be noted however that, as of this writing it is extremely difficult for any device other than a genuine Apple device running iOS to interoperate with another Apple iOS device and then only if developed by an authorized Apple developer. The foregoing is expressly done for the sake of data and device security. However in contrast to Apple, the Android operating system is built on open source Linux code and is more “open”. As a result, there are numerous third party applications that enable controlling one Android device, say a Samsung Galaxy smartphone, with any other device running the Android OS. This embodiment also assumes that CM 40 is able to request and obtain a secure wireless communications link with the smartphone as soon as the smartphone mounted within case 10 is turned on, or at the very latest when both case 10 and the smartphone are powered on. Bluetooth and WiFi communications need not be further described herein for purposes of this invention, but again this embodiment presumes that case 10 can gain control over the mobile productivity device at least to the extent of obtaining images from the productivity device's camera(s) and also to display images on the productivity device's screen. In this embodiment, when case 10 and the enclosed smartphone are held above and parallel to a surface or object to be scanned and the user presses the “on” switch on case 10, CM 40 similarly would issue commands to the smartphone requesting that the smartphone turn on its camera (or cameras if appropriate) and also requesting that the smartphone display be energized. Thereafter, CM 40 would transmit a data packet to the smartphone essentially comprising an optimal separation distance between case 10 and the surface or object over which case 10 together with the smartphone are being held. As described previously, for the components specified herein, for accessory case 10 that produces total irradiance of about 1-5 mW/cm², the optimal separation distance between case 10 and any surface to be sanitized is approximately 0.75-1.5 inches (1.9-3.8 cm). Upon receiving such data representing the optimal separation distance, the smartphone would then lock the focusing element of its camera at the corresponding focal length corresponding to the 0.75-1.5 inches (1.9-3.8 cm) optimal separation distance between accessory case 10 and the surface or object to be sanitized. After the smartphone determines from its camera(s) and the locked focal length that the smartphone disposed in case 10 is located at the optimal separation from the surface to be sanitized, the smartphone will generate a graphical representation of the effective sanitizing area and superimpose it on the smartphone's display providing the user with a visual indication of the effective sanitizing area as viewed against the surface or object being sanitized.

Still yet a further embodiment of accessory case 10 also assumes and depends on interoperation of components and subsystems within both accessory case 10 and whichever mobile productivity device disposed therein, for example a smartphone. As in the foregoing embodiment, this embodiment also necessarily requires a close degree of between the maker(s) of accessory case 10 and the mobile productivity device disposed therein, including but not limited to both case 10 and the mobile productivity device running substantially similar operating systems. As above, this embodiment also assumes that CM 40 would request a secure communications link with the smartphone as soon as the smartphone mounted within case 10. However, this embodiment would not require that the smartphone display be energized. Rather, CM 40 would transmit a data packet to the smartphone comprising an optimal separation distance between case 10 and the surface or object to be cleaned, or approximately 0.75-1.5 inches (1.9-3.8 cm). The smartphone would then monitor the focusing element to determine when the camera is positioned at the focal length corresponding to the optimal 0.75-1.5 inches separation distance between accessory case 10 and the surface or object to be sanitized. When the smartphone determines from its camera(s) that the smartphone disposed in case 10 is located at the optimal separation from the surface to be sanitized, the smartphone would issue an auditory alert sound, such as a beep or a chirp, through its built-in speaker. It is anticipated and expected that a user could even personalize the auditory alert sound to be personalized or uniquely different from any other system auditory alert such as incoming email or text.

Additionally, integrated distance measuring components and applications running on mobile productivity device 15 may equivalently serve as distance sensing module 15, for example the LIDAR-based three-dimensional scanner in Apple's iPhone 12 PRO. Third party applications that optically measure distance, height and width are also available for iOS- and Android-based productivity devices.

Exemplary Characteristics of Sanitizing Light

Light power emitted by any illumination source typically is expressed in watts (W), and irradiance or fluence is expressed in watts per unit area (W/d²) where d is a characteristic dimension. Additionally, for germicidal action, the total amount of UV radiation received over time, commonly referred to as dose, is important. The dose typically is expressed as irradiance multiplied by the time (t) of exposure in seconds, in other words power as expressed in millijoules per unit area (e.g., mJ/cm²), where 1 mJ is equivalent to 1 mW/sec).

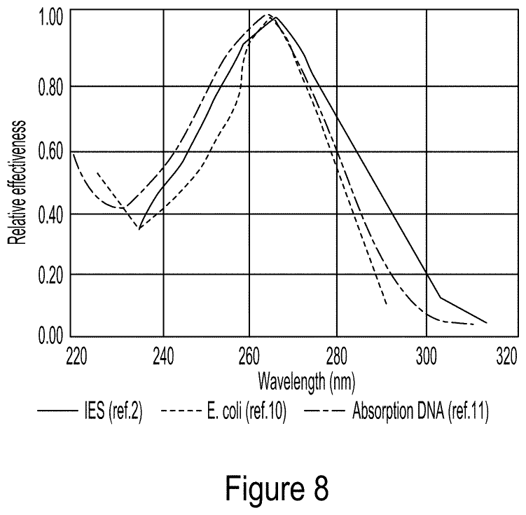

Referring now to FIG. 8, Philips Lighting NV, a long-time leader in industrial and municipal water sanitizing systems using UV, has studied the amount of disinfection as a function of wavelength. In FIG. 8, inactivation curves for three different bacterial species are shown as maximized at 265 nm with reductions on either side (Philips publication 3222 635 61801, November 2006). For wavelengths below 235 nm, germicidal action is not specified but it is reasonable to assume that it follows the DNA absorption curve. However, resistance of microorganisms to UV light is known to vary considerably, and the environment of the particular microorganism also can greatly affect the radiation dose needed to inactivate it. For example, water absorbs a part of the effective radiation depending on the concentration of contaminants in it. Additionally, iron salts in solution are known to absorb UV light. Table 2 in the cited Philips application note lists effective doses for a large variety of contaminant species.

UV Light Safety Considerations

The primary safety concern is that UV light of any degree may harm human skin and eye tissue. However, as reported in their April 2020 study, Buonanno, Welch, Shuryak and Brenner at Columbia University's Center for Radiological Research detected no adverse reaction to moderate to low dose exposure to 207-222 nm far UVC light while at the same time achieving essentially 100% inactivation of viral species.

According to Buonanno et al. in tests using human coronaviruses from subgroups alpha (HCoV-229E) and beta (HCoV-OC43), low doses of far UVC light (respectively 1.7 and 1.2 mJ/cm²) inactivated 99.9% of aerosolized alpha coronavirus 229E and beta coronavirus OC43. Based on these results for the beta HCoV-OC43 coronavirus, Buonanno et al. concluded that continuous far-UVC exposure in public locations at a recommended exposure limit (3 mJ/cm²/hour) should result in 99.9% viral inactivation in about 25 minutes. Doubling the far-UVC intensity would halve disinfection times while still maintaining safety. Human coronaviruses are believed to have similar genomic size, which is a key determinant of radiation sensitivity. Therefore it can be expected that far-UVC light will show comparable inactivation efficiency against other human coronaviruses, including SARS-CoV-2. According to Buonanno et al., the physical basis for this far-UVC approach is that this wavelength range (207-222 nm) is strongly absorbed by peptide bonds in proteins and other biomolecules. Therefore, the ability of far UVC light (below say 230 nm) to penetrate biological materials is very limited compared with, for example, 254 nm (or higher) conventional germicidal UVC light. On the other hand, this limited penetration is still much larger than the size of viruses and bacteria, so therefore far-UVC light is also as efficient in killing these pathogens as conventional germicidal UV light.

Additionally, German research institutes Ferdinand-Braun-Institut (FBH) and Technische Universitat Berlin (TUB) report having jointly developed UV LEDs emitting UV light that is safe to the skin. The LEDs created by FBH and TUB deliver far UV light at wavelengths around 230 nm while providing more than 1 mW output power. As reported by FBH, the 230 nm light does not penetrate into the living layers of the skin because of the high degree of absorption. Therefore it can be expected that human skin will not be adversely affected or will be harmed so little that natural healing responses will compensate for the effect. The first prototype reportedly was delivered to the Department of Dermatology at Charité-Universitätsmedizin Berlin for evaluation. Another LED reportedly will be evaluated in the near future by the Institute for Hygiene and Environmental Medicine of the University Medicine Center Greifswald for biocidal efficacy. The above work was funded by the German Federal Ministry of Education and Research (BMBF) as part of the consortium “Advanced UV for Life” within the Twenty20 program. Accordingly, far UVC LED devices with wavelengths below 230 nm can be expected to become commercially available in the foreseeable future.

In addition to careful UV LED selection, a child safety interlock mechanism also is incorporated within accessory case 10 to prevent youngsters or toddlers from inadvertently activating the UV light source and irradiating their eyes and skin. In one embodiment the child safety interlock may comprise a combination of operational controls that need to be activated simultaneously or in a specified sequence to turn on light source 20, or alternatively the equivalent of parental control settings via a user preferences menu accessed via CM 40. Furthermore, accessory case 10 has typical electrical safeguards and protections such as keyed connectors as well as shielding, ground planes and other measures to prevent improper power conditions (e.g., voltage and/or current surges) as well as electrostatic discharge within accessory case 10.

However, unlike broadband germicidal UV light, far UVC light cannot penetrate the human stratum corneum (the outer dead-cell skin layer), nor the ocular tear layer, or even the cytoplasm of individual human cells. Therefore, far UVC light cannot damage living cells in the human skin or the human eye, in contrast to the conventional germicidal UV light.

As a further precautionary measure against damaging skin or eye tissue, and with reference again to FIG. 3D, each embodiment may comprise a dosimeter module 96 coupled to the power source 30A-30B, control module 40 and data bus 70, wherein dosimeter module 96 measures dose of UVC light emitted by light source 20. Light intensity is known to decrease by d⁻³ when radiated into three-dimensional space (where d is the distance between light source 20 and whatever object or surface is being illuminated). Therefore it is important to position case 10 at the recommended separation distance from the surface or object to be sanitized. Hardware, that is to say electronic-based, UV dosimeters may comprise any of several different types such as radiometers, photodiodes, photocathodes sensitive to UV—visible radiation, and pyranometers. Several other types of UV dosimeters with greater sensitivity and accuracy are possible but require chemical or biological reagents to be externally utilized and then analyzed.

Importantly, dosimeter module 96 of this embodiment also is coupled to the distance-measuring device 95 via data bus 70 so as to permit calculation of the total dose delivered at the optimal separation distance, or alternatively to shut off light source 20 if/when a maximum dose is reached.

Additional Example Technical Features of UV LED Devices

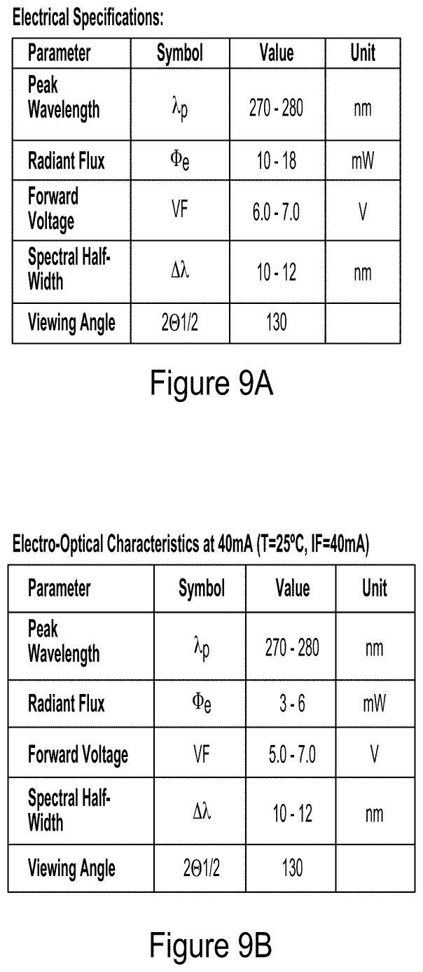

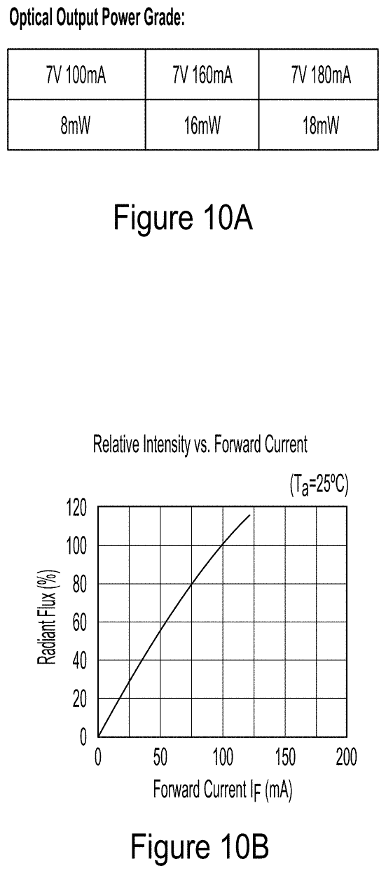

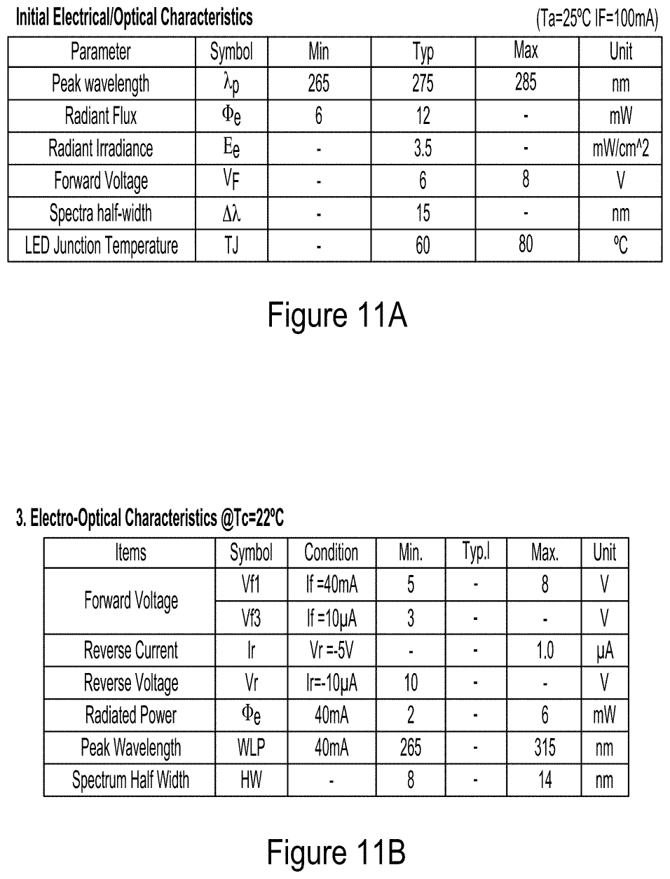

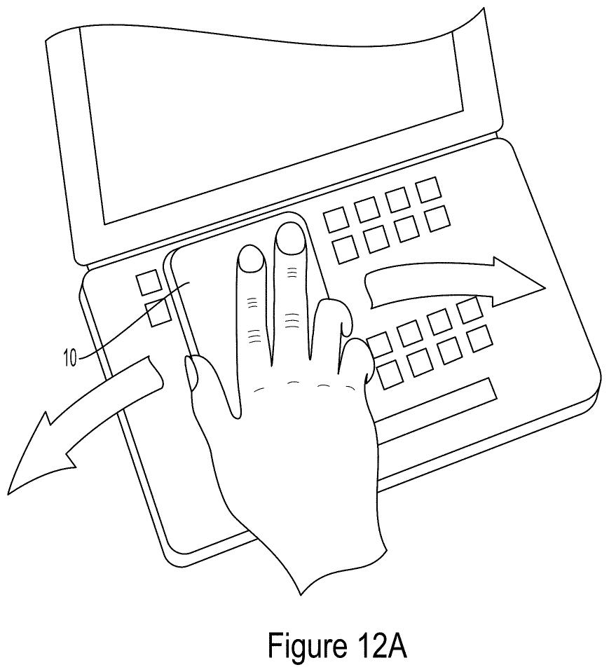

Technical specifications for UV LEDs are numerous and varied. Exemplary UV LED specifications are shown in FIGS. 9A, 9B, 10A, 10B, 11A and 11B, but are merely representative and not exhaustive. It should be understood that the number of LEDs as well as the power, wavelength and bandwidth of light emitted by the UV LED devices comprising light source 20 necessarily depends on the physical, electrical and optical characteristics (that is, voltage, current, wavelength) of the individual LEDs disposed in case 10, as well as nominal dose required for a specific sanitation application. Therefore, the actual number, spacing, arrangement and size of LEDs in light source 20 may vary significantly between manufacturers, comprising anywhere from a single LED device, to a horizontal row or vertical column of LED devices, to an array comprising multiple rows and columns of UV LEDs and emitting light comprising one or more wavelength ranges.

Example Method of Using the Portable Sanitizing Arrangement

Accessory case 10 enclosing a mobile productivity device such as a smartphone becomes a powerful disinfecting tool that conveniently goes wherever a user takes the smartphone. The method of using case 10 for disinfection/sanitation is similarly convenient and easy. A user simply turns on case 10 by pressing and holding on/off switch 105 for two seconds—which ensures that the user really intends to turn on case 10—after which CM 40 is initialized and executes the control executive stored within it. After case 10 is “on”, CM 40 issues a “light on” command as described above in connection with FIGS. 3B-3E that causes light source 20 to turn on. Thereafter, and as suggested by the illustration in FIG. 12A, the user simply moves or “scans” case 10 in a substantially horizontal plane above whatever surface or object that is to be sanitized in a moderately slow “step and repeat” motion while maintaining a relatively constant separation between case 10 and the surface or object being sanitized. The “step and repeat” scanning motion should continue until the recommended dose, that is exposure time at a given irradiance, has been delivered. With the arrangement of components as described herein, an effective UV sanitizing dose, that is irradiance over time, can be delivered in approximately thirty seconds.

As presently configured and also as shown in FIG. 12B, case 10 may be used within a laterally extending plane situated 0.75-1.5″ from the surface or object, or approximately 1.9-3.8 cm. For safety as well as operational convenience, it is anticipated that case 10 will be held and used in a generally horizontal attitude, also as shown in FIG. 12B. However, other angles of incidence of case 10 less than about 60° relative to surfaces or objects to be cleaned are acceptable as well so long as case 10 is moved within the laterally extending plane bounded by 0.75-1.5″ from the surface or object, or about 1.9-3.8 cm. As with any device generating and/or emitting UV light, caution should be exercised and case 10 should never be pointed at the skin or eyes of the user or any other person or animal. As configured and described herein, case 10 is only intended for disinfecting inanimate surfaces and objects that one encounters in any given day.

Although method operations, if any, were described in a specific order, it should be understood that other housekeeping operations may be performed in between operations, or operations may be adjusted so that they occur at slightly different times, or may be distributed in a system which allows the occurrence of the processing operations at various intervals associated with the processing, as long as the processing of the overlay operations are performed in the desired way.

The foregoing invention has been described in some detail for purposes of clarity of understanding. However it will be apparent that certain changes and modifications can be practiced within the scope of the appended claims. Accordingly, the present embodiments are to be considered as illustrative and not restrictive, and the invention is not to be limited to the details given herein, but may be modified within the scope and equivalents of the appended claims.

We claim:

1. An accessory case for a mobile productivity device, the accessory case, comprising:
a power source;
a control unit; and
an illumination source coupled to the power source and to the control unit and emitting light having a first wavelength range of about 200-280 nanometers for sanitizing microorganisms on a surface.

2. The accessory case of claim 1 wherein the illumination source comprises at least one light emitting diode.

3. The accessory case of claim 1 wherein the power source is coupled to a voltage booster circuit having an input voltage and an output voltage.

4. The accessory case of claim 3 wherein the voltage booster circuit increases the input voltage from about 3.7 volts to about 7.5 volts.

5. The accessory case of claim 1 wherein the illumination source produces an irradiance of about 1-5 milliwatts per square centimeter at a distance of about 2-4 centimeters from the surface.

6. The accessory case of claim 1 wherein the illumination source emits light having a second wavelength range of about 315-400 nanometers.

7. The accessory case of claim 6 wherein the illumination source emitting light having said second wavelength range comprises at least a second light emitting diode.

8. An accessory case for a mobile productivity device, the accessory case comprising a power source coupled to a control unit and to a first illumination source producing light having a first wavelength range of about 200-280 nanometers for sanitizing microorganisms on a surface.

9. The accessory case of claim 8 further comprising a second illumination source coupled to the power source and producing light having a second wavelength range of about 315-400 nanometers.

10. The accessory case of claim 9 wherein the first illumination source comprises at least one light emitting diode outputting a first ultraviolet wavelength range below about 280 nanometers and wherein the second illumination source comprises at least one light emitting diode outputting a second UV wavelength range above about 315 nanometers.

11. The accessory case of claim 8 wherein the power source is coupled to a voltage booster circuit having an input voltage and an output voltage.

12. The accessory case of claim 11 wherein the voltage booster circuit increases the input voltage from less than about 4 volts to an output voltage of greater than about 7 volts.

13. The accessory case of claim 12 wherein the first and second illumination sources produce an irradiance of about 1-5 milliwatts per square centimeter at a distance of about 1.9-3.8 centimeters from the surface.

14. A mobile productivity device accessory case for sanitizing surfaces, the accessory case comprising a case bottom portion and a case upper portion removeably joined and enclosing a control unit coupled to a power source producing a voltage and a current and an illumination source, said mobile productivity device accessory case producing light having a plurality of wavelength ranges wherein at least one of said wavelength ranges comprises wavelengths of about 200-280 nanometers and at least a second of said plurality of wavelength ranges comprises wavelengths of about 315-400 nanometers.

15. The mobile productivity device accessory case of claim 14 wherein the illumination source comprises a plurality of light emitting diodes.

16. The mobile productivity device accessory case of claim 15 wherein at least one of the plurality of light emitting diodes comprises a UVA light emitting diode.

17. The mobile productivity device accessory case of claim 15 wherein at least one of the plurality of light emitting diodes emits light at a wavelength less than about 230 nanometers.

18. The mobile productivity device accessory case of claim 14 further comprising a voltage booster circuit having an input and an output and coupled between the power source and the illumination source, the voltage booster circuit increasing the voltage received from the power source from less than about 4 volts at said input to greater than about 7 volts at its output.

19. The mobile productivity device accessory case of claim 18 producing an irradiance of greater than about 2500 μW/cm² across an area of about 0.25 square inches centered about a centrum of the illumination source and at a separation of about 1 inch between the illumination source and a surface to be sanitized.

20. The mobile productivity device accessory case of claim 19 producing an irradiance of not less than about 1000 μW/cm² across an area of about 4 square inches centered about the centrum of the illumination source and at a separation of about 1.5 inches between the illumination source and the surface to be sanitized.
